(19) United States
(12) Patent Application Publication
(10) Pub. No.: US 2008/0026448 A1
(43) Pub. Date: Jan. 31, 2008

(54) Production of HIV

(76) Inventors: Bjorn K. Lydersen, Encinitas, CA (US); Peter W. Lowry, King of Prussia, PA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/707,755

(22) Filed: Feb. 15, 2007

Related U.S. Application Data
(60) Provisional application No. 60/773,877, filed on Feb. 15, 2006.
(60) Provisional application No. 60/814,318, filed on Jun. 16, 2006.

(51) Int. Cl. C12N 7/00

(52) U.S. Cl. 435/235.1

(57) ABSTRACT

The present invention provides methods for producing HIV and HIV antigen by propagating HIV, in particular HIV-1, in cells grown in chemically defined, protein free medium. The invention also provides HIV antigen produced by such methods.

This application claims the benefit of priority of provisional application Ser. No. 60/773,877, filed Feb. 15, 2006, and claims the benefit of priority of provisional application Ser. No. 60/814,318, filed Jun. 16, 2006, each of which the entire contents is incorporated herein by reference.

### BACKGROUND OF THE INVENTION

This invention relates to Acquired Immunodeficiency Syndrome (AIDS) and, more specifically, to methods of producing HIV antigen for vaccine preparation.

More than 40 million people world wide are now infected with the human immunodeficiency virus (HIV), the virus responsible for AIDS. About 90% of HIV infected individuals live in developing countries, including sub-Saharan Africa and parts of South-East Asia, although the HIV epidemic is rapidly spreading throughout the world. Anti-viral therapeutic drugs that reduce viral burden and slow the progression to AIDS are available. However, these drugs are expensive and cumbersome for use in developing nations and also lead to mutant forms of the virus that are no longer killed by the antiviral drug. Thus, there remains an urgent need to develop effective preventative and therapeutic vaccines, known as immune based therapy (IBT) to curtail the global AIDS epidemic.

To date, HIV has proven a difficult target for effective vaccine development. Because of the propensity of HIV to rapidly mutate, there are now numerous strains predominating in different parts of the world with diverse epitopes. Additionally, in a particular infected individual, an HIV virus can escape from the control of the host immune system by developing mutations in an epitope. There remains a need to develop improved HIV vaccines that stimulate the immune system to recognize a broad spectrum of conserved epitopes, including epitopes from the p24 core antigen and key surface determinants as well. This need will require that such vaccines retain native structures of many viral antigens, such as a whole inactivated viral particle.

During the 1990's, more than 30 different candidate HIV-1 vaccines entered human clinical trials. These vaccines elicit various humoral and cellular immune responses, which differ in type and strength depending on the particular vaccine components. There remains a need to develop HIV vaccine compositions that strongly elicit the particular immune responses correlated with protection against HIV infection. The nature of protective HIV immune responses has been addressed through studies of individuals who have remained uninfected despite repeated exposure to HIV, or who have been infected with HIV for many years without developing AIDS. These studies have shown that CD4+ T helper cells correlate well with protection against HIV infection and subsequent disease progression. Besides antigen-specific CD4+ helper T cell responses, CD8+ cytotoxic T cell responses are considered important in preventing initial HIV infection and disease progression. During an effective anti-viral immune response, activated CD8+ T cells directly kill virus-infected cells and secrete cytokines with antiviral activity. Finally, a potent vaccine, especially a preventative vaccine, should stimulate humoral antibody responses, especially those able to neutralize a panel of primary isolates.

Compositions that elicit certain types of HIV-specific immune responses may not elicit other important protective responses. For example, Deml et al., Clin. Chem. Lab. Med. 37:199-204 (1999), describes a vaccine containing an HIV-1 gp160 envelope antigen or gene, an immunostimulatory DNA sequence and alum adjuvant, which, despite inducing an antigen-specific Th1-type cytokine response, was incapable of inducing an antigen-specific cytotoxic T lymphocyte response. Furthermore, a vaccine containing only envelope antigens would not be expected to induce an immune response against the more highly conserved core proteins of HIV. These vaccines likely do not mimic the native structures of the viral antigens in their native conformations.

For large scale production of HIV antigen for vaccine production, it would be advantageous to develop methods to facilitate production of HIV antigen for human clinical trials that gave maximal production of whole viral antigens with maximal maintenance of native viral proteins. HIV is propagated in mammalian cell culture, and such cultures generally use complex media that include proteins and other components, including serum generally obtained from fetal bovine. For human clinical trials, the HIV antigen must be purified away from such media components, particularly those that could elicit an immune response against non-HIV antigens. Therefore, it would be advantageous to develop methods to facilitate production of HIV antigen.

Thus, there exists a need for HIV immunogenic compositions and methods to facilitate production and purification of HIV antigen for vaccine production. The present invention satisfies this need and provides related advantages as well.

### SUMMARY OF INVENTION

The present invention provides methods for producing HIV and HIV antigen, including multiple native HIV antigens, by propagating HIV, in particular HIV-1, in cells grown in chemically defined, serum free and protein free medium.

The invention also provides HIV and HIV antigen produced by such methods.

### BRIEF DESCRIPTION OF THE DRAWINGS

FIG. 1 shows media screening. Cell growth and viral production is shown on Day 7 of culture.

FIG. 2 shows the effect of varying fructose/glucose concentrations on cell growth and productivity (Panel 1; Day 10 results).

FIG. 3 shows spent media analysis. Amino acid depletion is shown for spent medium (Panel SM).

FIG. 4 shows re-evaluation of potential improvements in culture conditions.

FIGS. 5A and 5B show a final comparison of cell growth (FIG. 5A) and viral production (FIG. 5B) in RPMI-1640 with 7% FBS, IS MAB-CD, and IRC T-VAX-CD media in roller bottle culture.

FIG. 6 shows analysis of purified HIV-1 antigen by Western immunoblot analysis. Lane 1: See Blue® molecular weight markers (Invitrogen; Carlsbad Calif.); Lane 2: HIV-1 grown in RPMI containing 7% FBS; Lane 3: HIV-1 grown in chemically defined, protein free medium. The total amount of protein loaded in lanes 2 and 3 was 1 μg each.

FIG. 7 shows analysis of purified HIV-1 antigen by sodium dodecyl sulfate polyacrylamide gel electrophoresis (SDS-PAGE). Lane 1: See Blue® molecular weight markers; Lane 2: HIV-1 grown in RPMI containing 7% FBS; Lane 3: HIV-1 grown in chemically defined, protein free serum free medium. The total amount of protein loaded in lanes 2 and 3 was 10 μg each.

### DETAILED DESCRIPTION OF THE INVENTION

The present invention provides methods of growing HIV in a protein free, serum free, chemically defined medium for production of HIV antigen for use as a component of an HIV vaccine. The methods of the invention allow production of HIV in a protein free medium, which facilitates purification of HIV for making HIV antigen as well as minimize potential immunogenic contaminants for production of an HIV vaccine. Various forms of an HIV antigen can be produced from HIV grown in protein free, chemically defined medium. A particularly useful form of an HIV antigen is whole inactivated HIV viral particles that are cultured and purified in a manner to maintain native conformation of the viral antigens. The invention provides an advantageous method of culturing, expanding, and manufacturing immunogenic HIV antigen, in particular whole inactivated viral particles that maintain a family of viral antigens in their native conformations for use in preventing HIV infection and for treating HIV infected individuals to delay their progression to AIDS. Such methods and compositions are advantageous since they are substantially free of animal components that could be potentially detrimental for inclusion in materials for clinical trials, such as an HIV vaccine. Contamination of vaccine material with animal components such as bovine albumin can potentially stimulate undesirable immune responses against the animal components, or can potentially contain undesirable prions, thereby making such a vaccine less desirable in clinical trials, and notably less desirable by the Federal Drug Administration (FDA) for commercial products. The methods and compositions of the present invention therefore advantageously provide animal component free material useful for an HIV vaccine.

The invention also provides methods for producing immunogenic HIV compositions containing an HIV antigen obtained from HIV grown in protein free, chemically defined medium. The invention additionally provides methods of immunizing a mammal with such compositions, or with the components of such compositions, so as to stimulate or enhance the immune response in the immunized mammal.

The invention provides methods for producing HIV in a chemically defined, protein free medium and HIV antigen obtained from HIV produced by such methods (see Examples I and III). In particular, the invention provides methods for producing HIV antigen by propagating HIV in a chemically defined, protein free medium containing fructose.

As disclosed herein, the addition of fructose to chemically defined, protein free medium was found to increase production of HIV grown in HUT-78 cells (see Examples I and III). Generally, a useful amount of fructose to include in a protein free, chemically defined media is about 1 gram/liter to about 8 grams/liter, in particular about 1 gram/liter, about 2 grams/liter, about 3 grams/liter, about 4 grams/liter, about 5 grams/liter, about 6 grams/liter, about 7 grams/liter, or about 8 grams/liter of fructose. As used herein, it is understood that a recited value is intended to include an amount about the value, and one skilled in the art readily understands the meaning of about, for example, about 1 gram/liter, and the like. The amount of glucose in the chemically defined, protein free medium can be correspondingly reduced, although generally at least a low amount of glucose is retained. In general, the addition of fructose to chemically defined, protein free medium provides improved growth, particularly with the reduction of glucose to low concentrations, for example, 1-2 grams/liter (see Examples I and III). In the case of a commercially available medium, the manufacturer can be instructed to reduce the amount of glucose based on the amount of fructose to be added, as desired. It is understood that derivatives of fructose that function similarly to fructose in increasing production of HIV can be used as well as other sugars having the same functional activity of increasing production of HIV, including structurally similar sugars. One skilled in the art can adjust fructose and glucose, or other components, to optimize growth conditions, as disclosed herein and described in Examples I and III.

A chemically defined medium can be modified to optimize cell culture conditions for HIV production, as described in Example III (see Table 2). For example, a commercially available medium, such as those described in Table 1 (see Example III), can be modified by one or more variables as described in Example III (see Table 2) to optimize growth of HIV. In particular, a commercially available medium such as IS MAB-CD™ can be modified to optimize cell culture conditions for HIV production (see Example III).

In one embodiment, the invention provides human immunodeficiency virus (HIV) in the presence of protein-free, chemically defined medium. In a particular embodiment, the HIV is HIV-1. The chemically defined medium can contain fructose. In a particular embodiment, the medium comprises 6 grams/liter of fructose, and can optionally further comprise 2 grams/liter of glucose, or other modifications that optimize HIV production, as disclosed herein. In another embodiment, the invention provides a whole inactivated human immunodeficiency virus (HIV).

The invention additionally provides a composition comprising HIV, wherein the HIV composition is substantially free of non-HIV proteins. In particular, the HIV composition is substantially free of albumin.

The invention also provides a composition comprising HIV substantially free of bovine albumin, wherein the HIV composition comprises less than 300 ng of bovine albumin per HIV antigen measured as a 10 μg/ml p24 dose. In a particular embodiment, the HIV composition comprises less than 60 ng of bovine albumin per HIV antigen measured as a 10 μg/ml p24 dose, for example, less than 50 ng, less than 40 ng, less than 30 ng, less than 20 ng, less than 10 ng, less than 5 ng, less than 2 ng, less than 1 ng, less than 0.5 ng, less than 0.1 ng, less than 0.05 ng, or less than 0.01 ng. In another embodiment, the invention provides a whole inactivated human immunodeficiency virus (HIV) substantially free of bovine albumin, or other HIV antigens as disclosed herein.

In another embodiment, the invention provides a composition comprising HIV free of animal components. As used herein, “free of animal components” means that the HIV was produced in a chemically defined medium in which no animal components were introduced. Such animal components are typically introduced into a medium in the form of serum, so such an HIV composition is produced in a serum free medium. Furthermore, such an HIV composition can be produced in a protein free medium. An “animal component” is considered to be derived from a non-human animal. In the case of HIV produced in culture, typically a human cell line is used, such as HUT-78 or other appropriate cells, as disclosed herein. As HIV buds from such a cell, proteins, carbohydrates, lipids, and the like, from the human cell line may become a component of the HIV, and such human-derived components are not considered to be an “animal component” as used herein.

In another embodiment, the invention provides a method for producing HIV by growing HIV infected cells, for example, HUT-78 cells or other suitable cells, in a protein-free, chemically defined medium. Such a protein-free, chemically defined medium can contain fructose. Furthermore, the invention provides methods of producing whole inactivated human immunodeficiency virus (HIV), wherein the whole inactivated HIV is produced from HIV infected HUT-78 cells grown in a protein-free, chemically defined medium, for example, a medium containing fructose. If desired, the whole inactivated HIV can be depleted of outer envelope protein gp120. The invention additionally provides a composition comprising HIV produced by the methods of the invention.

As disclosed herein, the invention provides a method of producing HIV in a Wave bioreactor (see Examples). In a particular embodiment, the invention provides a method for producing HIV by growing HIV infected cells in a protein-free, chemically defined medium in a Wave bioreactor. As disclosed herein, a Wave bioreactor allows cell culture in pre-sterile plastic bags as single use bioreactors. The Wave Bioreactor eliminates the need for cleaning, sterilization, and associated validation of the cleaning and sterilization. The cell culture bags reduce the risk of contamination due to equipment malfunction or operator error that can occur typically with traditional bioreactors such as stirred tanks, spinners, and hollow-fiber systems. This can be particular useful with production of infectious HIV. In addition, a Wave bioreactor is advantageous for potentially growing HIV cultures for vaccine production in places where traditional cell culture methods or fermentors requiring specialized equipment would not be economically feasible. Additionally, Wave bioreactors can be advantageously utilized with protein free medium, as disclosed herein. The presence of proteins and particularly serum can cause foaming in the Wave bioreactor generated by the wave-like action. The use of a serum free or protein free medium generates less foaming and can be beneficial when growing a culture in a Wave bioreactor.

Furthermore, the use of serum free or protein free chemically defined medium can facilitate downstream processing, particularly on ultrafiltration steps where high concentrations of protein in the medium can slow the filtration process, thereby leading to more efficient protein clearance. Therefore, the use of serum free, protein free chemically defined media can facilitate downstream processing of HIV harvested from cell cultures.

Methods of purifying HIV from cultured cells are well known to those skilled in the art (see U.S. Pat. Nos. 5,661,023 and 6,080,571). An exemplary production and purification method for HIV in a bioreactor is described in Examples I and IIII.

While exemplified herein with HIV, it is understood that the methods disclosed herein can similarly be applied to other viruses for growth in chemically defined, protein free medium. For example, other viruses, including other retroviruses, can be grown in chemically defined, protein free medium. Similarly, although exemplified with the HUT-78 cell line, the methods of the invention can similarly be applied to other cell lines suitable for propagation of a virus of interest, in particular HIV.

Advantageously, the compositions of the invention containing HIV antigen produced from HIV grown in chemically defined, protein free medium can also induce HIV specific CD4 T helper cells and CD8+ T cells, yielding potent Th1 immune responses against a broad spectrum of HIV epitopes, providing a strong HIV-specific cytotoxic T lymphocyte response. Thus, the immunogenic compositions of the invention are useful for preventing HIV infection and/or slowing progression to AIDS in infected individuals. The compositions containing HIV antigen produced from HIV grown in chemically defined, protein free medium and methods of using such compositions can be used to elicit potent Th1 cellular and humoral immune responses specific for conserved HIV epitopes, elicit HIV-specific CD4 T helper cells, HIV-specific cytotoxic T lymphocyte activity, stimulate production of chemokines and cyotokines such as β-chemokines, interferon-γ, interleukin 2 (IL2), interleukin 7 (IL7), interleukin 15 (IL15), alpha-defensin, and the like, and increase memory cells. Such vaccines can be administered via various routes of administration. Such vaccines can be used to prevent maternal transmission of HIV, for vaccination of newborns, children and high-risk individuals, and for vaccination of infected individuals. Such vaccines can optionally include immunomers or an immunostimulatory sequence (ISS) to enhance an immune response against the HIV antigen. Such vaccines can also be used in combination with other HIV therapies, including antiretroviral therapy (ART) with various combinations of nuclease and protease inhibitors and agents to block viral entry, such as T20 (see Baldwin et al., Curr. Med. Chem. 10:1633-1642 (2003)).

As used herein, the term “HIV” refers to all forms, subtypes and variations, including clades, of the HIV virus, including HIV-1 and HIV-2, and is synonymous with the older terms HTLVIII and LAV. Various cell lines capable of propagating HIV or permanently infected with the HIV virus have been developed and deposited with the ATCC, including HuT 78 cells and the HuT 78 derivative H9, as well as those having accession numbers CCL 214, TIB 161, CRL 1552 and CRL 8543, which are described in U.S. Pat. No. 4,725,669 and Gallo, Scientific American 256:46 (1987). It is understood that a cell line can be infected with a desired HIV, including one or more specific clades, and used in methods of the invention for production of an HIV vacccine, as disclosed herein.

As used herein, the term “whole inactivated HIV virus” refers to an intact, inactivated HIV virus. An inactivated HIV refers to a virus that cannot infect and/or replicate. A whole inactivated HIV virus generally maintains native structure of viral antigens to maintain immunogenicity and stimulate immune responses to native virus. If desired, a whole inactivated virus can be modified to deplete proteins from the virus, in particular outer envelope proteins. As used herein, the term “outer envelope protein” refers to that portion of the membrane glycoprotein of a retrovirus which protrudes beyond the membrane, as opposed to the transmembrane protein, gp41. For example, a whole inactivated virus can be depleted of monomeric gp120 while maintaining native trimeric gp160, composed of gp120 and gp41. Thus, a particularly useful whole inactivated virus is one largely depleted of monomeric gp120 but retains native trimeric gp160.

As used herein, the term “HIV virus devoid of outer envelope proteins” or “HIV virus depleted of outer envelope proteins” refers to a preparation of HIV particles or HIV gene products in which the outer envelope protein gp120 has been substantially depleted, but contains the more genetically conserved parts of the virus (for example, p24 and gp41). Such a depleted virus can contain gp160, which includes trimeric gp120 and gp41. An HIV devoid or depleted of the outer envelope protein gp120 is also referred to herein as REMUNE™. It is understood that such a preparation can have substantially all of gp120 removed but can also contain low levels of gp120 and still be considered an HIV virus devoid or depleted of outer envelope proteins.

As used herein, the term “HIV p24 antigen” refers to the gene product of the gag region of HIV, characterized as having an apparent relative molecular weight of about 24,000 daltons designated p24. The term “HIV p24 antigen” also refers to modifications and fragments of p24 having the immunological activity of p24. Those skilled in the art can determine appropriate modifications of p24, such as additions, deletions or substitutions of natural amino acids or amino acid analogs, that serve, for example, to increase its stability or bioavailability or facilitate its purification, without destroying its immunological activity. Likewise, those skilled in the art can determine appropriate fragments of p24 having the immunological activity of p24. An immunologically active fragment of p24 can have from 6 residues from the polypeptide up to the full length polypeptide minus one amino acid. Other HIV antigens encoded by other HIV gene products can include fragments or modifications similar to those described above for the HIV p24 antigen. Other exemplary HIV antigens include, for example, gp41, nef, and the like.

As used herein, an “immunomer” refers to an oligonucleotide comprising two smaller oligonucleotides linked at their 3′ ends, resulting in an oligonucleotide having two 5′ ends. The two smaller oligonucleotides of the immunomer can be identical or non-identical sequences and/or lengths, but generally are identical. In addition to its immunostimulatory activity, an immunomer contains a 3′-3′ linkage and therefore has no free 3′ end, thus increasing resistance to nuclease digestion. Thus, in one embodiment, an immunomer comprises two identical oligonucleotides linked via their 3′ ends. An immunomer can also include modified bases. Immunomers and methods of making immunomers are described, for example, in Kandimalla et al., Bioorg. Med. Chem. 9:807-813 (2001); Yu et al., Nucl. Acids Res. 30:4460-4469 (2002); Yu et al., Bioorg. Med. Chem. 11:459-464 (2003); Bhagat et al., Biochem. Biophys. Res. Comm. 300:853-861 (2003); and Yu et al., Biochem. Biophys. Res. Comm. 297:83-90 (2002); Yu et al., Nucl. Acids Res. 30:1613-1619 (2002); Yu et al., J. Med. Chem. 45:4540-4548 (2002); Kandimalla et al., Bioconjugate Chem. 13:966-974 (2002); Yu et al., Bioorganic Med. Chem. Lett. 10:2585-2588 (2000); Agrawal and Kandimalla, Trends Mol. Med. 8:114-121 (2002); WO 98/55495, each of which is incorporated herein by reference. Such immunomers can have more potent immunostimulatory activity than immunostimulatory sequences containing CpG. An immunomer enhances the immune response in a mammal when administered in combination with an antigen. An immunomer can be a CpG immunomer or CpG-free immunomer, as discussed below. The use of immunomers with an HIV antigen are described in U.S. publication 2005/0196411 and PCT publication WO 2005/021726, each of which is incorporated herein by reference.

As used herein, a “CpG immunomer” refers to an immunomer, as described above, that specifically contains a CpG motif. Thus, a CpG immunomer is an oligonucleotide comprising two identical or non-identical smaller oligonucleotides, where at least one of the smaller oligonucleotides contains at least one CpG motif. As used herein, a “CpG-free immunomer” refers to an immunomer that specifically excludes a CpG motif. Thus, a CpG-free immunomer is an oligonucleotide comprising two identical or non-identical smaller oligonucleotides, where neither of the smaller oligonucleotides contains a CpG motif.

An immunomer can contain either natural or modified nucleotides or natural or unnatural nucleotide linkages. Modifications known in the art, include, for example, modifications of the 3′OH or 5′OH group, modifications of the nucleotide base, modifications of the sugar component, and modifications of the phosphate group. An unnatural nucleotide linkage can be, for example, a phosphorothioate linkage in place of a phosphodiester linkage, which increases the resistance of the nucleic acid molecule to nuclease degradation. Various modifications and linkages are described, for example, in PCT publication WO 98/55495.

As used herein, the term “immunostimulatory sequence” or “ISS” refers to a nucleotide sequence containing an unmethylated CpG motif that is capable of enhancing the immune response in a mammal when administered in combination with an antigen. Immunostimulatory sequences are described, for example, in PCT publication WO 98/55495.

As used herein, the term “adjuvant” refers to a substance which, when added to an immunogenic agent, nonspecifically enhances or potentiates an immune response to the agent in the recipient host upon exposure to the mixture. Adjuvants can include, for example, oil-in-water emulsions, water-in oil emulsions, alum (aluminum salts), liposomes and microparticles, such as polysytrene, starch, polyphosphazene and polylactide/polyglycosides. Adjuvants can also include, for example, squalene mixtures (SAF-I), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875. For veterinary use and for production of antibodies in animals, mitogenic components of Freund's adjuvant (both complete and incomplete) can be used. In humans, Incomplete Freund's Adjuvant (IFA) is a particularly useful adjuvant. Various appropriate adjuvants are well known in the art and are reviewed, for example, by Warren and Chedid, CRC Critical Reviews in Immunology 8:83 (1988).

As used herein, “AIDS” refers to the symptomatic phase of HIV infection, and includes both Acquired Immune Deficiency Syndrome (commonly known as AIDS) and “ARC,” or AIDS-Related Complex, as described by Adler, Brit. Med. J. 294: 1145 (1987). The immunological and clinical manifestations of AIDS are well known in the art and include, for example, opportunistic infections and cancers resulting from immune deficiency.

As used herein, the term “inhibiting AIDS” refers to a beneficial prophylactic or therapeutic effect of an HIV immunogenic composition in relation to HIV infection or AIDS symptoms. Such beneficial effects include, for example, preventing or delaying initial infection of an individual exposed to HIV; reducing viral burden in an individual infected with HIV; prolonging the asymptomatic phase of HIV infection; maintaining low viral loads in HIV infected patients whose virus levels have been lowered via anti-retroviral therapy (ART); increasing levels of CD4 T cells or lessening the decrease in CD4 T cells, both HIV-1 specific and non-specific, in drug naive patients and in patients treated with ART, increasing overall health or quality of life in an individual with AIDS; and prolonging life expectancy of an individual with AIDS. A clinician can compare the effect of immunization with the patient's condition prior to treatment, or with the expected condition of an untreated patient, to determine whether the treatment is effective in inhibiting AIDS.

As used herein, the term “enhances,” with respect to an immune response against HIV antigen is intended to mean that an immunogenic composition elicits a greater immune response than does a composition containing HIV antigen alone. In the case where the immunogenic composition contains the three components HIV antigen, immunomer or ISS and adjuvant, the immunogenic composition elicits a greater immune response than does a composition containing any two of the three components of the immunogenic composition, administered in the same amounts and following the same immunization schedule. The components of the immunogenic compositions of the invention can act in synergy. An enhanced immune response can be, for example, increased production of chemokines and/or cytokines that promote memory cells, an increase in memory cells, an increase in IgG2b production, in increase in cytotoxic T lymphocyte activity, an increase in β-chemokine or IL15 production, and the like. As an example of an enhanced immune response, the immunogenic compositions of the invention can increase production of γ-interferon by both CD4 cells (helper function) and CD8 cells (cytotoxic T lymphocytes; CTLs).

As used herein, the term “β-chemokine” refers to a member of a class of small, chemoattractive polypeptides that includes RANTES, macrophage inflammatory protein-1β (MIP-1β) and macrophage inflammatory protein-1α (MIP-1α). The physical and functional properties of β-chemokines are well known in the art. In the case of enhanced β-chemokine production, the β-chemokine production can be “HIV-specific β-chemokine production,” which refers to production of a β-chemokine in response to stimulation of T cells with an HIV antigen. Alternatively, or additionally, the β-chemokine production that is enhanced can be “non-specific βchemokine production,” which refers to production of a 1-chemokine in the absence of stimulation of T cells with an HIV antigen.

As used herein, the term “kit” refers to components packaged or marked for use together. For example, a kit can contain an HIV antigen from virus grown in chemically defined, protein free medium, optionally an immunomer or ISS and optionally an adjuvant, for example, in three separate containers. Alternatively, a kit can contain any two components in one container, and a third component and any additional components in one or more separate containers. Optionally, a kit further contains instructions for combining the components so as to formulate an immunogenic composition suitable for administration to a mammal.

The invention also provides an immunogenic composition containing an HIV antigen produced from HIV grown in chemically defined, protein free medium, optionally an immunomer or ISS, and further optionally an adjuvant. The immunogenic composition enhances the immune response in a mammal administered the composition. In one embodiment, the immunogenic composition enhances an HIV-specific cytotoxic T lymphocyte (CTL) response in a mammal. In another embodiment, the immunogenic composition enhances HIV-specific CD4+ helper T cells.

In one embodiment, the HIV antigen in the immunogenic composition is a whole inactivated HIV virus obtained from HIV grown in chemically defined, protein free medium, which can be prepared by methods known in the art. For example, HIV virus can be prepared by culture from a specimen of peripheral blood of infected individuals. In an exemplary method of culturing HIV virus, mononuclear cells from peripheral blood (for example, lymphocytes) can be obtained by layering a specimen of heparinized venous blood over a Ficoll-Hypaque density gradient and centrifuging the specimen. The mononuclear cells are then collected, activated, as with phytohemagglutinin for two to three days, and cultured in an appropriate medium, preferably supplemented with interleukin 2 (IL2). The virus can be detected either by an assay for reverse transcriptase, by an antigen capture assay for p24, by immunofluorescence or by electron microscopy to detect the presence of viral particles in cells, all of which are methods well known to those skilled in the art. In addition, HIV virus can be grown in a cell line, as disclosed herein (see Examples I and III).

Methods for isolating whole-killed HIV particles are described, for example, in Richieri et al., Vaccine 16:119-129 (1998), and U.S. Pat. Nos. 5,661,023 and 5,256,767. In one embodiment, the HIV virus is an HZ321 isolate from an individual infected in Zaire in 1976, which is described in Choi et al., AIDS Res. Hum. Retroviruses 13:357-361 (1997).

Various methods are known in the art for rendering a virus non-infectious (see, for example Hanson, MEDICAL VIROLOGY II (1983), de la Maza and Peterson, eds., Elsevier,). For example, the virus can be inactivated by treatment with chemicals or by physical conditions such as heat or irradiation. The virus is treated with an agent or agents that maintain the immunogenic properties of the virus. For example, the virus can be treated with beta-propiolactone or gamma radiation, or both beta-propiolactone and gamma radiation, at dosages and for times sufficient to inactivate the virus for use in a vaccine.

In another embodiment, the HIV antigen in the immunogenic composition is a whole inactivated HIV virus devoid or depleted of outer envelope proteins, which can be prepared by methods known in the art. In order to prepare whole inactivated virus devoid or depleted of outer envelope proteins, the isolated virus is treated so as to remove at least some of the outer envelope proteins. Such removal is preferably accomplished by repeated freezing and thawing of the virus in conjunction with physical methods which cause the swelling and contraction of the viral particles, although other physical or non-physical methods, such as sonication, can also be employed alone or in combination.

In yet another embodiment, the HIV antigen in the immunogenic composition is one or more substantially purified gene products of HIV, for example, purified from HIV grown in chemically defined, protein free medium, as disclosed herein. Such gene products include those products encoded by the gag genes (p55, p39, p24, p17 and p15), the pol genes (p66/p51 and p31-34) and the transmembrane glycoprotein gp41; and the nef protein. These gene products may be used alone or in combination with other HIV antigens. The HIV antigen can also be peptide fragments of HIV gene products that illicit an immune response.

The substantially purified gene product of HIV can be a substantially purified HIV p24 antigen or other HIV antigens and gene products. p24, as well as other HIV antigens, can be substantially purified from the virus by biochemical methods known in the art, or can be produced by cloning and expressing the appropriate gene in a host organism such as bacterial, fungal or mammalian cells, by methods well known in the art. Alternatively, p24 antigen, or a modification or fragment thereof that retains the immunological activity of p24, as well as other HIV antigens or modifications or fragments thereof, can be synthesized, using methods well known in the art, such as automated peptide synthesis. If more than one substantially purified HIV gene product is used, one gene product can be produced by purification from HIV grown in chemically defined, protein free medium, another can be produced by recombinant techniques and/or another by chemical synthesis. Determination of whether a modification or fragment of p24 retains the immunological activity of p24, or other viral antigens retain their respective immunological activity, can be made, for example, by their ability to stimulate proliferation in vitro of previously immunized PBMCs as analyzed by conventional lymphocyte proliferation assays (LPA) known in the art (see U.S. publication 2005/0196411 and WO 2005/021726), by immunizing a mammal and comparing the immune responses so generated, or testing the ability of the modification or fragment to compete with p24 for binding to a p24 antibody, or other HIV antigens to their respective antibodies.

In still another embodiment, the HIV antigen in the immunogenic composition is a substantially purified gene product of a protease defective HIV (see U.S. Pat. Nos. 6,328,976 and 6,557,296). Such an HIV antigen can be obtained from a protease defective HIV grown in chemically defined, protein free or serum free medium, as disclosed herein.

The replication process for HIV-1 has an error rate of about one per 5-10 base pairs. Since the entire viral genome is just under 10,000 base pairs, this results in an error rate of about one base pair per replication cycle. This high mutation rate contributes to extensive variability of the viruses inside any one person and an even wider variability across populations. This variability has resulted in three HIV-1 variants being described and around 10 subspecies of virus called “clades.” These distinctions are based on the structure of the envelope proteins, which are especially variable. The M (for major) variant is by far the most prevalent world wide. Within the M variant are clades A, B, C, D, E, F, G H, I, J and K, with clades A through E representing the vast majority of infections globally. Clades A, C and D are dominant in Africa. Clade B is the most prevalent in Europe, North and South America and Southeast Asia. Clades E and C are dominant in Asia. These clades differ from on another by as much as 35%.

There are two important results from the very high mutation rate of HIV-1 that have profound consequences for the epidemic. First, the high mutation rate is one of the mechanisms that allows the virus to escape from control by drug therapies. These new viruses represent resistant strains. The high mutation rate also allows the virus to escape the patient's immune system by altering the structures that are recognized by immune components. An added consequence of this extensive variability is that the virus can also escape from control by vaccines, and vaccines based on envelope proteins will likely be non-effective.

The greatest variation in structure is seen in the envelope proteins gp120 and gp41. Less variation is seen in the various internal proteins. REMUNE™ is an immunogen that is made from the whole virus depleted or devoid of its gp120 proteins but contains most of the highly conserved epitopes of the HIV-1 virus. A whole inactivated virus preparation depleted of gp120, as with REMUNE™, can be depleted of monomeric gp120 but retain gp160, containing gp120 and gp41. This and other whole inactivated virus are particularly useful since native structure is retained to allow generation of an immune response against native HIV. Both the number of these epitopes and their lower incidence of mutation mean that an HIV virus devoid or depleted of outer envelope proteins stimulates the immune responses that have a greater chance of success within individuals. In addition, the HuT-78 cell line was purposely infected with a very early strain of HIV virus containing both clades A and G for conserved antigens, which have been retained across most variations in clades seen worldwide, and this HuT 78 HIV infected cell line provided virus used as HIV antigen. Thus, the use of an HIV virus with multiple early clades that is also devoid or depleted of outer envelope proteins for immunization can be effective across clades by providing conserved antigens that can be recognized by most patients.

For an HIV antigen combined with an immunomer or ISS, the HIV antigen and an immunomer or ISS can be mixed together, or can be conjugated by either a covalent or non-covalent linkage. Methods of conjugating antigens and nucleic acid molecules are known in the art, and exemplary methods are described in PCT publication WO 98/55495.

Those skilled in the art can readily determine whether a particular HIV antigen, immunomer, or combination thereof is effective in stimulating or enhancing a desired immune response in a particular mammal by immunizing a mammal of the same species, or a species known in the art to exhibit similar immune responses, with a composition containing a particular immunomer. A variety of assays known in the art can then be used to characterize and compare the characteristics of the immune responses induced. For example, an optimized immunomer to include in an immunogenic composition for administration to a human can be determined in either a human or a non-human primate, such as a baboon, chimpanzee, macaque or monkey by evaluating its immune activity, for example, by LPA, ELISPOT, and/or ratios of IgG1/G2 antibody produced.

The immunogenic compositions of the invention can further contain an adjuvant, such as an adjuvant demonstrated to be safe in humans. An exemplary adjuvant is Incomplete Freund's Adjuvant (IFA). Another exemplary adjuvant contains mycobacterium cell wall components and monophosphoryl lipid A, such as the commercially available adjuvant DETOX™. Another exemplary adjuvant is alum. The preparation and formulation of adjuvants in immunogenic compositions are well known in the art.

Optionally, the immunogenic compositions of the invention can contain or be formulated together with other pharmaceutically acceptable ingredients, including sterile water or physiologically buffered saline. A pharmaceutically acceptable ingredient can be any compound that acts, for example, to stabilize, solubilize, emulsify, buffer or maintain sterility of the immunogenic composition, which is compatible with administration to a mammal and does not render the immunogenic composition ineffective for its intended purpose. Such ingredients and their uses are well known in the art.

The invention also provides kits containing an HIV antigen purified from HIV grown in chemically defined, protein free medium, optionally an immunomer or ISS, and optionally an adjuvant. The components of the kit, when combined, produce an immunogenic composition which enhances an immune response in a mammal. The components of the kit can be combined ex vivo to produce an immunogenic composition containing an HIV antigen, an immunomer or ISS and optionally an adjuvant. Alternatively, any two components can be combined ex vivo, and administered with a third component, such that an immunogenic composition forms in vivo. For example, an HIV antigen can be emulsified in, dissolved in, mixed with, or adsorbed to an adjuvant and injected into a mammal, preceded or followed by injection of immunomer. Likewise, each component of the kit can be administered separately. Those skilled in the art understand that there are various methods of combining and administering an HIV antigen, optionally an immunomer or ISS, and optionally an adjuvant, so as to stimulate or enhance the immune response in a mammal.

Immunogenic compositions of the invention containing adjuvant and/or immunomer or ISS can enhance the strength and potency of the immune response to an HIV immunogen, thereby enhancing therapeutic and/or preventative efficacy of a vaccine. An enhanced immune response can be, for example, increased production of HIV-1 specific CD4+ helper T cells, chemokines and/or cytokines, an increase in memory cells, an increase in overall antibody production and more specifically in the ratio of IgG2b production, an increase in cytotoxic T lymphocyte activity, an increase in β-chemokine or IL15 production, and the like. Thus, the immunogenic compositions of the invention can be used to enhance TH1 cytokine profile (high IFNγ, high IgG2/IgG1 ratios). The components of the immunogenic compositions of the invention can act in synergy. For example, the immunogenic compositions of the invention can enhance β-chemokine production by eliciting production of a higher concentration of β-chemokine than would be expected by adding the effects of pairwise combinations of components of the immunogenic composition.

Memory cells are needed for maintaining long term immunity following the initial acute state of infection. During the contraction phase following an initial acute stage of infection, a significant amount of the immune cells induced against the infectious agent are destroyed by apoptosis, with only the surviving cells remaining able to become memory cells. Therefore, protecting HIV-specific CD4 and CD8 T cells from apoptosis promotes an increase in both HIV-specific CD4 helper and CD8 CTL memory cells. The immunogenic compositions of the invention can be used to increase memory cells, thereby promoting long term helper functions and cell-mediated immunity. The immunogenic compositions of the invention can be used to increase the number of memory cells by decreasing apotosis or by stimulating factors that promote survival of memory cells.

The immunogenic compositions of the invention containing an HIV antigen obtained from cells grown in chemically defined, protein free medium, optionally an immunomer or ISS and optionally an adjuvant can be used to shift a TH2 to a TH1 response, thereby increasing cell-mediated immune responses, including a stronger CD8+ response. Thus, the immunogenic compositions of the invention can be used to strengthen the immune response in a patient, who otherwise is only responding weakly, and convert the response to cell-mediated immunity. The immunogenic compositions of the invention can thus be used to increase the strength and duration of an immune response in a patient that would have responded weakly to a similar HIV antigen as that used in the immunogenic composition.

An immunogenic composition of the invention can be used to enhance an immune response, for example, enhanced β-chemokine and/or IL15, IFN, IL2, TNFα production, increased HIV-specific CD4 helper cells, IgG2b antibody production, HIV-specific cytotoxic T lymphocyte (CTL) production, IFNγ production by CD4+ cells and CD8 T cells, and the like, in a mammal administered the composition. As described in U.S. Pat. No. 6,737,066 and WO 00/67787, each of which is incorporated herein by reference, and in U.S. publication 2005/0196411 and WO 2005/021726, production of the β-chemokine RANTES can be detected and quantitated using an ELISA assay of supernatants of T cells (such as lymph node cells or peripheral blood cells) from mammals administered the composition. In order to determine antigen-specific β-chemokine production, T cells from an immunized mammal can be stimulated with HIV antigen in combination with antigen-presenting thymocytes, and the β-chemokine levels measured in the supernatant. In order to determine non-specific β-chemokine production, either T cell supernatant or a blood or plasma sample from an immunized mammal can be assayed. Similarly, production of other β-chemokines, such as MIP-1α and MIP-1β, can be detected and quantitated using commercially available ELISA assays, according to the manufacturer's instructions. Methods of measuring cytokine production, including inteferon, IL15, IL2, TNFα, IL10 and IL7, by ELISPOT, ELISA, or intracellular cytokine staining are well known to those skilled in the art (see, for example, Robbins et al., AIDS 17:1121-1126 (2003)).

An immunogenic composition of the invention can further be capable of enhancing HIV-specific IgG2b antibody production in a mammal administered the composition. High levels of IgG2b antibodies, which are associated with a Th1 type response, are correlated with protection against HIV infection and progression to AIDS. Thus, the invention provides compositions that can increase a TH1 response. An immunogenic composition of the invention can further be capable of enhancing HIV-specific cytotoxic T lymphocyte (CTL) responses in a mammal administered the composition. An immunogenic composition of the invention can increase IFN-γ production by both CD4+ T cells and CD8+ T cells.

IFN-γ production by CD4+ T cells is characterized as a classic CD4 helper response important to cell-mediated immunity. CD4+ T cells producing both IFN and IL2 may be most effective. IFN-γ production by CD8+ T cells is representative of a cytotoxic T lymphocyte (CTL) response, and is highly correlated with cytolytic activity. Cells producing both IFN and TNFα may be most effective. CTL activity is an important component of an effective prophylactic or therapeutic anti-HIV immune response. Methods of determining whether a CTL response is stimulated or enhanced following administration of an immunogenic composition of the invention are well known in the art, and include cytolytic assays and LPA assays (described, for example, in Deml et al. supra (1999);), and ELISA and ELISPOT assays for CD8-specific IFN-γ production (see U.S. Pat. No. 6,737,066, WO 00/67787, U.S. publication 2005/0196411 and WO 2005/021726), intracellular staining and FACS analysis using a myriad of antibodies against cell surface markers.

The invention also provides a method of immunizing an individual. The method consists of simulating or enhancing the immune response in an individual by administering to a mammal an immunogenic composition containing an HIV antigen prepared from HIV grown in chemically defined, protein free medium, optionally an immunomer or ISS, and optionally an adjuvant. The components of the immunogenic composition can be administered in any order or combination, such that the immunogenic composition is formed ex vivo or in vivo.

In a particular embodiment, the HIV antigen, optional immunomer or ISS and optional adjuvant are administered simultaneously or at about the same time, in about the same site. However, administering the components within several minutes or several hours of each other can also be effective in providing an immunogenic composition that stimulates or enhances an immune response. Additionally, administering the components at different sites in the mammal can also be effective in providing an immunogenic composition that stimulates or enhances an immune response. One skilled in the art can readily determine a suitable time and location to separately administer components, that is, the HIV antigen, optional immunomer or ISS and optional adjuvant components, to provide a sufficient immune response by administering the separate components at various times and locations and measuring the immune response. The immunogenic composition can also be administered multiple times, if desired, for example, 2 or more, 3 or more, 4 or more, 5 or more, 6 or more, 7 or more, 8 or more, 9 or more, or 10 or more, or any desired number of times to stimulate or enhance an HIV-specific immune response.

The immunogenic compositions of the invention can be administered to a human to inhibit AIDS, such as by preventing initial infection of an individual exposed to HIV, reducing viral burden in an individual infected with HIV, prolonging the asymptomatic phase of HIV infection, increasing overall health or quality of life in an individual with AIDS, or prolonging life expectency of an individual with AIDS. Administration to a mammal of an immunogenic composition containing an HIV antigen, optionally an isolated nucleic acid molecule containing an immunomer or ISS, and optionally an adjuvant stimulates immune responses correlated with protection against HIV infection and progression to AIDS (see U.S. publication 2005/0196411 and WO 2005/021726).

In particular, when an immunogenic composition containing HIV antigen, optionally an immunomer or ISS and/or adjuvant, the immunogenic compositions stimulate or enhance the immune response more effectively than would be expected by combination of any of the individual components or, in a three component composition containing HIV antigen, immunomer or ISS and adjuvant, any two components of the immunogenic compositions. Additionally, the immunogenic compositions promote strong Th1 type immune responses, including both Th1 type cytokines (for example, IFN-γ) and Th1 type antibody isotypes (for example, IgG2b). Thus, the immunogenic compositions of the invention will be effective as vaccines to prevent HIV infection when administered to seronegative individuals, and to reduce viral burden, prolong the asymptomatic phase of infection, and positively affect the health or lifespan of a seropositive individual.

Individuals who have been exposed to the HIV virus usually express in their serum certain antibodies specific for HIV. Such individuals are termed “seropositive” for HIV, in contrast to individuals who are “seronegative.” The presence of HIV specific antibodies can be determined by commercially available assay systems. At the present time, serological tests to detect the presence of antibodies to the virus are the most widely used method for determining infection. Such methods can, however, result in both false negatives, as where an individual has contracted the virus but not yet mounted an immune response, and in false positives, as where a fetus may acquire the antibodies, but not the virus from the mother. Where serological tests provide an indication of infection, it may be necessary to consider all those who test seropositive as in fact, being infected. Further, certain of those individuals who are found to be seronegative may in fact be treated as being infected if certain other indications of infection, such as contact with a known carrier, are satisfied.

The immunogenic compositions of the invention can be administered to an individual who is HIV seronegative or seropositive. In a seropositive individual, it may be desirable to administer the composition as part of a treatment regimen that includes treatment with anti-viral agents, such as protease inhibitors. Anti-viral agents and their uses in treatment regimens are well known in the art, and an appropriate regimen for a particular individual can be determined by a skilled clinician.

As described in U.S. Pat. No. 6,737,066, WO 00/67787, U.S. publication 2005/0196411 and WO 2005/021726, administration of the immunogenic compositions of the invention to a primate fetus or to a primate neonate results in the generation of a strong anti-HIV immune response, indicating that the immune systems of fetuses and infants are capable of mounting an immune response to such compositions which should protect the child from HIV infection or progression to AIDS. Accordingly, the immunogenic compositions of the invention can be administered to an HIV-infected pregnant mother to prevent HIV transmission to the fetus, or to a fetus, an infant, a child or an adult as either a prophylactic or therapeutic vaccine.

The dose of the immunogenic composition, or components thereof, to be administered in the methods of the invention is selected so as to be effective in stimulating the desired immune responses. Generally, an immunogenic composition formulated for a single administration contains between about 1 to 200 μg of protein antigen. An immunogenic composition generally contains about 100 μg of protein antigen for administration to a primate, such as a human. As described in U.S. Pat. No. 6,737,066, WO 00/67787, U.S. publication 2005/0196411 and PCT publication WO 2005/021726, about 100 μg of HIV antigen in an immunogenic composition elicits a strong immune response in a primate. About 10 μg of HIV antigen is suitable for administration to a rodent. One skilled in the art can readily determine a suitable amount of HIV antigen to include in an immunogenic composition of the invention sufficient to stimulate an immune response.

The immunogenic compositions of the invention can further contain from about 5 μg to about 100 μg of an immunomer, and can contain up to 10 mg of immunomer, if desired, as described in U.S. publication 2005/0196411 and PCT publication WO 2005/021726. As described previously in U.S. Pat. No. 6,737,066 and WO 00/67787, a ratio of at least 5:1 by weight of nucleic acid molecule to HIV antigen was more effective than lower ratios for eliciting immune responses. One skilled in the art can readily determine an appropriate or optimized ratio of immunomer or ISS to HIV antigen for eliciting an immune response. For example, the ratio can be varied and the immune response measured by methods disclosed herein to determine a suitable or optimized ratio of immunomer or ISS to HIV antigen. In rodents, an effective amount of an immunomer in an immunogenic composition is from 5 μg to greater than 50 μg, such as about 100 μg. In primates, about 500 μg of an immunomer is suitable in an immunogenic composition. Those skilled in the art can readily determine an appropriate amount of immunomer to elicit a desired immune response.

As with all immunogenic compositions, the immunologically effective amounts are determined empirically, but can be based, for example, on immunologically effective amounts in animal models, such as rodents and non-human primates. Factors to be considered include the antigenicity, the formulation (for example, volume, type of adjuvant), the route of administration, the number of immunizing doses to be administered, the physical condition, weight and age of the individual, and the like. Such factors are well known in the vaccine art and it is well within the skill of immunologists to make such determinations without undue experimentation.

The immunogenic compositions of the invention can be administered locally or systemically by any method known in the art, including, but not limited to, intramuscular, intradermal, intravenous, subcutaneous, intraperitoneal, intranasal, oral or other mucosal routes. The immunogenic compositions can be administered in a suitable, nontoxic pharmaceutical carrier, or can be formulated in microcapsules or as a sustained release implant. The immunogenic compositions of the invention can be administered multiple times, if desired, in order to sustain the desired immune response. The appropriate route, formulation and immunization schedule can be determined by those skilled in the art.

It is understood that modifications which do not substantially affect the activity the various embodiments of this invention are also provided within the definition of the invention provided herein. Accordingly, the following examples are intended to illustrate but not limit the present invention.

### EXAMPLE I

### Development of a Protein Free, Chemically Defined Cell Culture Medium for the Production of HIV from HUT-78 Cells

One approach to the development of an HIV vaccine is to utilize whole, inactivated HIV particles formulated with one or more adjuvants. This approach is currently being tested as a therapeutic vaccine in clinical trials. The virus is currently produced in stirred tank fermenters by chronically infected HUT-78 cells, a human T-cell lymphoma line, utilizing a culture medium consisting of RPMI-1640 medium supplemented with fetal bovine serum as the culture medium. The goal of this project was to develop a chemically defined, protein-free medium for the production process that would eliminate the presence of all animal-derived components, improve virus yield, provide reliable growth and scale-up of the HIV-1 infected cells, and maintain equivalence of the virus in terms of its performance as an immunological stimulant against HIV.

The first phase of the project was to screen over 20 commercially available serum-free media designed for a variety of cells. Results showed that about one-third of the media formulas provided for at least equivalent cell density and virus yield (measured by p24 ELISA) compared to the serum-containing control medium. However, most of these formulas were unable to maintain reliable growth when cells were passaged repeatedly. One formula, IS MAB-CD™ (Irvine Scientific, Irvine Calif.), was able to provide satisfactory long-term growth. This formula provided for high cell densities, but virus yields were significantly lower than in many of the other media. Due to the importance of reliable cell passaging, this formula was chosen as the basis for further optimization work.

A total of 18 panels of test media were prepared using the IS MAB-CD™ base medium in order to evaluate the effect of varying the concentrations of key ingredients, individually, or in combination. Several panels resulted in “dose-response” curves, indicating that virus yield could be improved by altering the concentration of certain components. These panels included potassium and phosphate concentration, osmolality, lipids, metabolic intermediates, and most significantly the partial replacement of glucose with fructose. The reduction of glucose to 1-2 g/L and the addition of fructose to 2-6 g/L resulted in higher cell densities, greatly increased longevity of the cultures, and a 3-fold increase in virus yield relative to medium with high glucose alone (6 g/L). This effect may in part be due to the maintenance of the culture pH above 7.0 in the presence of low glucose. The concentrations of amino acids in spent media were also analyzed, and it was found that serine, methionine and tryptophan were significantly depleted. A 1.5- to 2-fold increase in the initial concentration of these amino acids improved cell growth significantly but did not change the amount of viral particles in the supernatant.

The final stage of the project utilized the IS MAB-CD medium with fructose as the basal medium, and the other promising changes were evaluated individually and in combination. Results showed that only one additional change was found to increase the virus yield further, an increase in the concentration of a mix of eight metabolic intermediates.

These results show that (1) reliable cell growth was obtained with a chemically defined medium without serum or any protein constituents in the medium; (2) virus yield could be improved 2-fold over that obtained with serum and 3-fold over the serum-free medium originally chosen; and (3) the most significant factor in the improvement of virus yield was the inclusion of fructose as a partial alternative to glucose. These results, initially obtained utilizing stationary T-flasks, were confirmed in roller bottles and WAVE cultures.

### EXAMPLE II

### Method of Production of HIV

This example describes production and purification of HIV in chemically defined, protein free medium.

The following steps are performed to produce and purify HIV grown in chemically defined, protein free medium. The purification method is similar to that described in U.S. Pat. Nos. 5,661,023 and 6,080,571, each of which is incorporated herein by reference.

An overview of the steps for production and purification of HIV in chemically defined, protein free medium is as follows:
Step (1), initiate culture from master working cell bank (MWCB)
Step (2), cell culture of HIV-1/HuT78 in serum-free, protein-free, chemically-defined media. Culture parameters are optimized for bioreactors.
Step (3), clarify culture supernatant by filtration. Filtration conditions can be optimized.
Step (4), treat with β-propiolactone (βPL) at 4° C.
Step (5), perform concentration & diafiltration. Conditions can be optimized.
Step (6), apply concentrate to TMAE Ion Exchange column (TMAE FRACTOGEL, Merck (Damstadt Germany)(see U.S. Pat. Nos. 5,661,023 and 6,080,571).
Step (7), TMAE column elution (see U.S. Pat. Nos. 5,661,023 and 6,080,571).
Step (8), six-fold dilution in 50 mM Na₂HPO₄.
Step (9), apply to Q Sepharose™ fast flow (QSFF) ion exchange column (see U.S. Pat. Nos. 5,661,023 and 6,080,571).
Step (10), QSFF column elution.
Step (11), concentrate and diafilter.
Step (12), ultracentrifuge, resuspend, and freeze.
Step (13), perform ⁶⁰Co gamma Irradiation.
Step (14), purify inactivated HIV-1 Antigen and dilute to a standard protein concentration (1.2 mg/mL total protein) and freeze.

An exemplary purification is described in more detail below for cells grown in a bioreactor. Suitable modifications for other cell culture configurations, for example, roller bottles or WAVE bioreactor (Wave Biotech, Bridgewater N.J.), can be made to optimize growth in other cell culture configurations. It is understood that one skilled in the art can modify the procedure described below to optimize growth and purification conditions, as desired.

Cells from the Manufacturer's Working Cell Bank (MWCB) are grown in chemically defined, protein free medium and expanded in T flasks followed by spinners or roller bottles, progressing from T25 to increasing sized T flasks and to roller bottles, to provide inoculum for the bioreactor. At the time of seeding, at a minimal density of 2×10⁵ cells/mL, cells are in exponential growth with viabilities above 65%. Exponential growth of cell cultures minimizes the lag phase in the bioreactor to within five days following initial seeding. When the cells start to grow, 24 hours after seeding, and achieve a density between 5×10⁵ to 9×10⁵, a continuous supply of fresh medium is provided, and spent medium is removed on a continuous basis. The rate of medium exchange commences at approximately 0.5 reactor volume/day and is increased to 4 reactor volumes/day when cell density reaches about 2 to about 4×10⁶ cells/mL. In this continuous state of perfusion, cell densities of about 8×10⁶ are achieved, which is a consequence of delivering nutrients and removing waste products on a continuous basis. Therefore, perfusion typically achieves about 10 to about 20 fold higher cell density than the average daily cell density obtained in spinners, where cells are grown in a batch mode.

A bioreactor can be used having an external hollow fiber filtration device with a piping module that recirculates cells back into the reactor from the retentate side of the filter membrane while spent medium is withdrawn from the permeate side. Use of a hollow fiber filter, with a 0.1 micron pore size, for the removal of waste products results in almost complete retention of viral particles within the bioreactor, thus facilitating removal of the viral particles in the daily harvest along with cells. A feed pump supplies fresh medium, which maintains constant volume under control of a level sensor. A perfusion rate of 4 reactor volumes/day generally supports a density of about 6 to about 8×10⁷ cells/mL, at which point cell growth approaches a stationary phase. This condition will result in reduction of culture viability and/or growth rate. To maintain cells with an optimal growth rate, daily harvesting of reactor fluid is performed daily to “cut-back” density, for example to about 3 to about 6×10⁶ cells/mL, which is roughly equivalent to harvesting one third of a reactor volume/day. This also maintains high viability and reduces cell debris by the removal of dead cells. Most importantly, this daily harvest volume from the inside of the reactor permits removal of viral particles.

The initial step in the recovery and purification of viral particles from the daily harvest uses removal of cells and cellular debris using micro-filtration procedures. This involves use of a dead-end filtration device utilizing a membrane with a pore size in the range of about 0.65 to about 0.8 microns and up to about 5.0 microns. This procedure serves to separate the cells and cell debris from the viral particles.

This procedure typically involves adding beta-propiolactone (βPL) to clarified harvest at a final concentration of 0.25-0.5 mL βPL per liter of supernatant, followed by incubation at 4° C. for 18-24 hours and then at 37° C. for 5 hours to inactivate the βPL by hydrolysis. The pool is subsequently held at 4° C. for 0-6 days until further processing. This inactivation step serves to chemically reduce the infectivity of the viral particles by alkylating the various structural constituents of the virus such as lipids, proteins, nucleic acids, and the like. While the βPL inactivation step is effective at inactivating virus, the material is still handled as infectious and is treated as such for subsequent processing.

The βPL treated material is allowed to reach ambient temperature and is concentrated 10 to 20 fold by tangential flow ultrafiltration using a 300,000 MW cutoff membrane. The concentrated pool is then diafiltered against 5-10 volumes of phosphate buffered saline (PBS), pH 7.5. The pore size of the membrane used in this step serves to retain HIV particles while removing greater than 90% of relatively small molecules in the permeate.

The pH of the concentrated and diafiltered material is adjusted to pH 7.5 and is loaded onto a column containing TMAE FRACTOGEL™ resin at a linear flow rate of 50 cm/hr. The column is washed with approximately 3-5 column volumes of 0.5 M NaCl, pH 6.5. Bound product is then eluted using 1.0 M NaCl, pH 6.5, and the entire peak absorbing at 280 nm is collected. The column is subsequently sanitized following a 1 to 2 column volume wash with 1.0N NaOH. The product contained in the 1.0 M NaCl elution fraction is diluted six fold to reduce the salt concentration to within the range 0.1-0.2 M NaCl and pH 6.5-7.5. It is then applied to a Q-SEPHAROSE FAST FLOW™ column (Pharmacia/GE Healthcare, Piscataway N.J.) which contains approximately one half the amount of resin compared to the TMAE FRACTOGEL™ column at a linear flow rate of 100 cm/hr. The column is washed with 3-5 column volumes of 0.6 M NaCl, pH 6.5. The bound product is eluted again using the 1.0 M NaCl buffer.

The 1.0 M NaCl fraction from the Q-SEPHAROSE FAST FLOW™ column is concentrated approximately 5-fold using an ultrafiltration plate and frame device containing a 100,000-300,000 MW cut-off membrane. The concentrate is then diafiltered against PBS, pH 7.5, to reduce the salt concentration. To minimize product loss in dead space following concentration, the equipment is flushed with 1 dead space volume of saline (0.9% NaCl) and the wash is added to the diafiltered fraction. The material is frozen at −70° C., and is subjected to cobalt irradiation (for example, 2 to 4.5 megarads or 4 to 6 megarads for approximately 24 hours), which serves as a final viral inactivation step.

At this stage the preformulated product is diluted with 0.9% saline for injection to achieve the required concentration of antigen (generally p 24 concentration, for example, 100-200 μg p24 antigen per ml), and is optionally aseptically mixed with an equal volume of Incomplete Freund's Adjuvant using a shaker device for approximately 15-20 minutes. Additional components can be added, as desired.

The above-described production method in a bioreactor can be adapted for use in a Wave Bioreactor. A Wave Bioreactor allows cell culture in pre-sterile plastic bags as single use bioreactors. The Wave Bioreactor can incorporate feedback controls for pH, dissolved oxygen and other critical culture parameters. The Wave Bioreactor eliminates the need for cleaning, sterilization, and associated validation of the cleaning and sterilization. The cell culture bags reduce the risk of contamination due to equipment malfunction or operator error that can occur typically with traditional bioreactors such as stirred tanks, spinners, and hollow-fiber systems. This can be particular useful with production of infectious HIV.

The cell culture bag is filled with chemically defined, protein free medium and inoculated with MWCB, generally from T-flask or roller bottle seed cultures propagated from the MWCB. The bags are inflated to form a rigid gas-impermeable chamber. The bags are placed on a Wave Bioreactor rocking platform and rocked to induce a wave-like motion. The gentle wave motion provides oxygenation and mixing with minimal shear forces. Air is continually passed through the headspace utilizing sterilizing filters provided on each bag. On completion of cultivation, the cell culture is harvested and processed as described above.

### EXAMPLE III

### Development of a Serum-Free, Chemically Defined Cell Culture Medium for the Production of HIV from HUT-78 Cells

One approach to the development of an HIV vaccine is to utilize whole, inactivated HIV formulated with one or more adjuvants. This approach is currently being tested as a therapeutic vaccine in clinical trials. The virus is currently produced in stirred tank fermenters by chronically infected HUT-78 cells, a human T-cell lymphoma line, utilizing a culture medium consisting of RPMI-1640 medium supplemented with fetal bovine serum. The goal of this project was to develop a chemically defined, protein-free medium for the production process that would eliminate the presence of all animal-derived components, improve virus yield, provide for reliable growth and scale-up of the cells, and maintain equivalence of the virus in terms of its performance as an immunological stimulant against HIV.

The first step in the project was to screen over 20 commercially available serum-free media designed for a variety of cells. Results showed that about one-third of the media formulas provided for at least equivalent cell density and virus yield (measured by p24 ELISA) compared to the serum-containing control medium. However, most of these formulas were not able to maintain reliable growth when cells were passaged repeatedly. One formula, IS MAB-CD, was able to provide for completely satisfactory long-term growth. This formula provided for high cell densities, but virus yields were significantly lower than in many of the other media. Due to the importance of reliable cell passaging, this formula was chosen as the basis for future optimization work.

A total of 18 panels of test media were prepared using the ISMAB-CD base medium in order to evaluate the effect of varying the concentrations of key ingredients, individually or in combination. Several panels resulted in “dose-response” curves indicating that virus yield could be improved by altering the concentration of certain ingredients. These panels included potassium and phosphate concentration, osmolality, lipids, metabolic intermediates, and most significantly the partial replacement of glucose with fructose. The reduction of glucose to 1-2 g/L and the addition of fructose to the medium resulted in higher cell densities, greatly increased longevity of the culture, and a three-fold increase in virus yield compared to medium with high glucose alone (6 g/L). This effect may be due to the maintenance of the culture pH above 7.0 in the presence of fructose. The concentrations of amino acids in spent media were also analyzed, and it was found that serine, methionine and tryptophan were significantly depleted. A 1.5-2× increase in the initial concentrations of these amino acids improved cell growth significantly, but did not change the amount of viral particles in the supernatant.

The final stage of the project utilized the ISMAB-CD medium with fructose as the basal medium, and the other promising changes were evaluated individually and in combination. However, only one additional change was found to be of value in increasing the virus yield; this was an increase in the concentration of a mix of metabolic intermediates. The overall conclusions from the project were; 1) reliable cell growth was obtained with a chemically defined medium without serum or any protein constituents in the medium, 2) virus yield was improved two-fold over that obtained with serum and three-fold over the serum-free medium originally chosen, and 3) the most significant factor in the improvement of virus yield was the inclusion of fructose as a partial alternative to glucose. These results, initially obtained utilizing stationary T-flasks, were confirmed in roller bottles and WAVE cultures.

Briefly, in more detail, one approach to the development of an HIV vaccine is to utilize whole, inactivated HIV formulated with one or more adjuvants. This approach is being evaluated as a therapeutic vaccine in clinical trials, utilizing Freund's incomplete adjuvant and an immunomodulatory oligonucleotide (HYB2055) (Trabattoni et. al., Vaccine 24:1470-1477 (2006)). The intent of this vaccine is to stimulate both cell and antibody mediated responses to a broad array of HIV antigens. The HIV-1 virus used in this vaccine is the HZ321 strain. It is produced by HUT-78 cells (a human T cell lymphoma cell line) which are chronically infected with the virus. The current production process is carried out in 250 L stirred tank fermenters, utilizing RPMI-1640 cell culture medium supplemented with 7% fetal bovine serum. The clarified cell culture supernatant is treated with beta-propiolactone to chemically inactivate the virus, and then purified by several steps including ultrafiltration, ion exchange chromatography and ultracentrifugation, prior to a second inactivation step with gamma irradiation. The goal of this project was to develop a chemically defined, protein-free cell culture medium for the production process that would eliminate all animal-derived components, improve viral yield, provide reliable and consistent cell growth from the seed stocks through to full-scale production, and maintain equivalence of the virus in terms of its performance as an immunological stimulant against HIV. Further details are described below.

I. Screening of Serum-Free Media Formulations

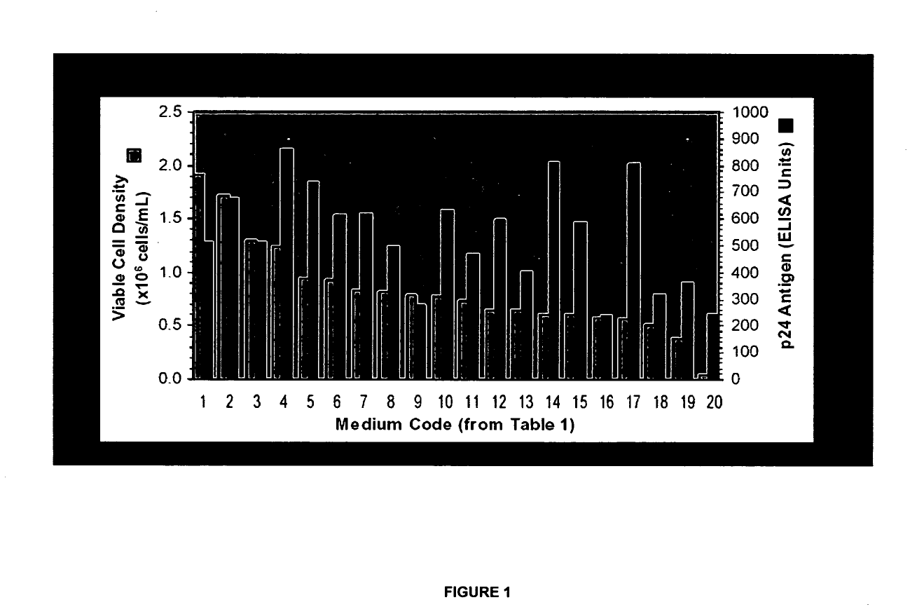

The first step in the project was to determine if currently available serum-free media formulas could provide for growth and virus production from the cell line. It was determined that FBS could not be completely removed from RPMI-1640, but a serum-free D-MEM formula with insulin, transferrin and albumin permitted passaging of the cells without FBS. Cells adapted to this medium were utilized in the screening of over twenty serum-free formulas, including those listed in Table 1. These formulas were chosen to represent a wide range of formulas in terms of the cells for which they were developed, the supplements used (proteins, lipids and hydrolysates), and the companies which supplied them. A summary of the key growth and virus yield data is shown in FIG. 1. The cell densities and the kinetics of cell growth varied significantly, as did the yield of virus, and the amount of virus produced per cell. There was no consistent pattern in terms of which cell type the media had been developed for, or which supplements seemed to be important.

Three formulas, IS MAB-CD, Gibco CD 293, and Ex-Cell Sp2/0, were chosen for further evaluation based on their ability to either support high density cell growth (IS MAB-CD) or a high specific productivity with adequate growth (CD 293 and Ex-Cell 293). Cells were passaged in each of these media for three weeks and then re-evaluated for virus production in T flasks and roller bottles. The IS MAB-CD provided for significantly better results in passaging and yielded a comparable level of virus production. Based on its growth promoting attributes, IS MAB-CD was chosen as the basal medium for further optimization work, as described in the next section.

TABLE 1
Serum-Free Media Screened for Growth and Virus Protection

| | | | Media | |
|---|---|---|---|---|
| | | | Composition/ | |
| Code | Product | Company | Additions | Cell Type |
| 1 | IS NS0 | Irvine | with lipids | NS0 |
| | | Scientific | | |
| 2 | IS MAB-CD | Irvine | Chemically | Hybridoma |
| | | Scientific | defined | |
| 3 | 293 SFMII | Gibco | protein | 293 |
| 4 | CD 293 | Gibco | Chemically | 293 |
| | | | defined | |
| 5 | CD Hybridoma | Gibco | Chemically | Hybridoma |
| | | | defined | |
| 6 | Ex-Cell 325 | JRH | soy hydrolysate | CHO |
| 7 | Hybridoma SF | Sigma | protein | Hybridoma |
| 8 | Hybridoma SFM | Gibco | protein | Hybridoma |
| 9 | Ex-Cell 293 | JRH | protein | 293 |
| 10 | Ex-Cell Sp2/0 | JRH | Chemically | Sp2/0 |
| | | | defined | |
| 11 | Ex-Cell 302 | JRH | soy hydrolysate | CHO |
| 12 | RMPI-1640 (+5% | HyClone | Fetal Bovine | B-cells |
| | FBS) | | Serum | |
| 13 | Advanced D-MEM | Gibco | protein | |
| 14 | IR-02 | D. Wyatt | | custom |
| 15 | HyQ-CCMI | HyClone | | |
| 16 | MegaCell RPMI | Sigman | | B-cells |
| 17 | IR-05 | D. Wyatt | | custom |
| 18 | SF/PF Hybridoma | Sigma | | Hybridoma |
| 19 | IMAT | IRC | protein and lipid | HUT-78 |
| 20 | Cell MAB | BD | | Hybridoma |

II. Optimization of the Basal Medium

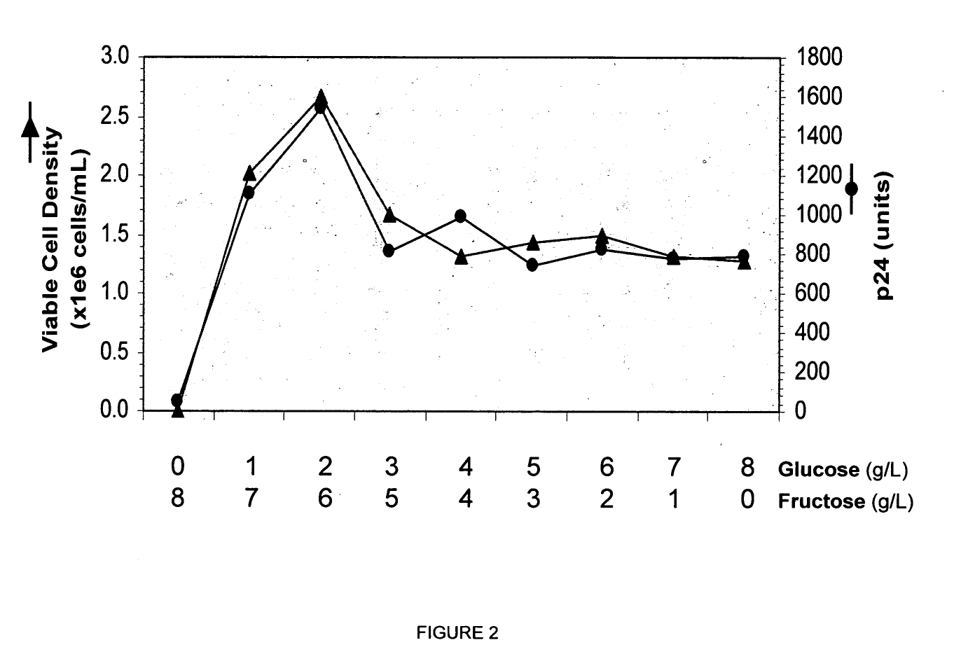

The main approach to the optimization of the IS MAB-CD basal medium was to create test “panels” of media in which one or a related group of ingredients was varied. Table 2 shows the panels (prepared by Irvine Scientific) that were tested, and the over-all conclusions reached from each panel. The first panel, which evaluated the partial substitution of fructose for glucose, was by far the most significant in the improvement of virus yield. There have been reports that virus yields after infection can be raised by the maintenance of a higher pH, and one way of increasing culture pH without feedback control is to substitute fructose (or certain other carbohydrates) for glucose (Low and Harbour. Growth kinetics of hybridoma cells; (2) the effects of varying energy source concentrations. Develop. Biol. Standard. 60 (1985), 73-79.) As FIG. 2 illustrates, the reduction of glucose and the addition of fructose resulted in at least a 50% increase in virus yield and cell growth. Subsequent experiments showed that low glucose alone (2 g/L) did not result in improved growth or yield.

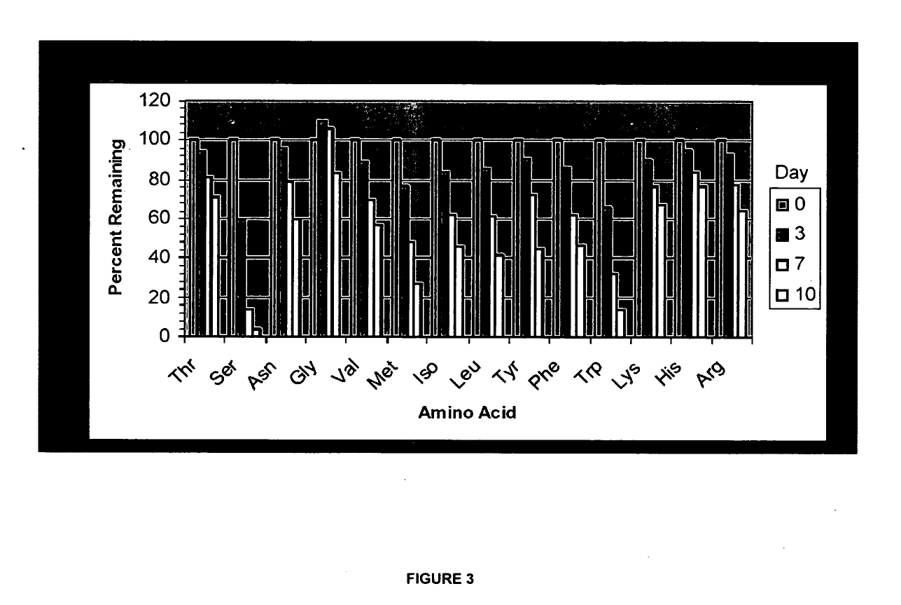

The results from the other panels are summarized in Table 2. Several variables were found to have different optimum levels than those present in the IS MAB-CD medium, with improvements in viral yield varying from 10-50%. An analysis of amino acids in “spent media” (with fructose) was performed as well. Six amino acids did not decrease in concentration, including cystine. The remaining 14 amino acids were depleted to varying extents as shown in FIG. 3. The concentrations of three amino acids (serine, methionine and tryptophan) were found to be significantly depleted. When the initial concentration of these 3 amino acids was raised by 1.5-2×, the growth and viability of cells after day 7 was improved significantly.

TABLE 2
Summary of Medium Optimization Panels and Results.

| | | Conclusion | Approximate |
|---|---|---|---|
| Panel | Name | (Optimal Range) | Improvement |
| 1 | Glucose/Fructose | 6:2 glucose/fructose ratio | 50% |
| | Ratio | | |
| 2 | Osmolality | 350mOsm/Kg | 10-30% |
| 3 | Potassium | 15 mM | 25% |
| 4 | Glutamine | 2-15 mM | — |
| 5 | Glutamine Feed | no benefit | — |
| 6 | Pluronic F-68 | 10× lower | 30% |
| 7 | Cation DoE | 1.5× K & Ca | 10-30% |
| 8 | Nucleosides | 0.5-6 mg/L | 50% |
| 9 | Reducing Agents | 1-2× | — |
| 11 | Phosphate | 0.6× | 0-50% |
| 12 | Cation DoE | 1× | — |
| | Verification | | |
| 13 | Trace Metals | 0.5-2× | — |
| 14 | Iron | 0.5-5× | — |
| 16 | Fructose | ≧4 g/L | 50% |
| 17 | Vitamins | 0.5-1× | — |
| 18 | Groups DoE | 1.75× Metabolic | 20% |
| | | Intermediates | |
| SM | Spent Media Analysis | 3 amino acids increased | |
| | | 1.5-2× | |

III. Final Evaluation of Changes to the Basal Medium

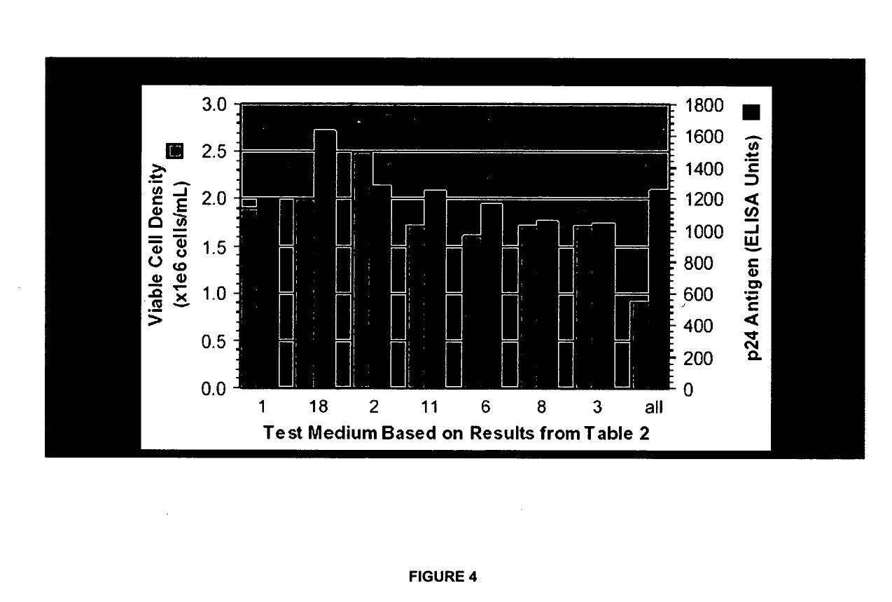

The potential changes to the basal medium identified in the testing of the 18 panels summarized in Table 2 were re-evaluated in a single panel, utilizing IS MAB-CD with 6 g/L fructose and 2 g/L glucose. The results are summarized in FIG. 4. The only change that appeared to have a major impact on virus production in this experiment was the increase in the amount of the “metabolic intermediates”. Additional testing confirmed that the increase in metabolic intermediates improved viral yield, and that the increase in the depleted amino acids improved longevity of the culture. It was decided that the IS MAB-CD formula would be changed to include; a) 6 g/L fructose, 2 g/L glucose, b) 1.5× the concentration of the metabolic intermediates, and c) 1.5-2× increase in the three amino acids found to be significantly depleted.

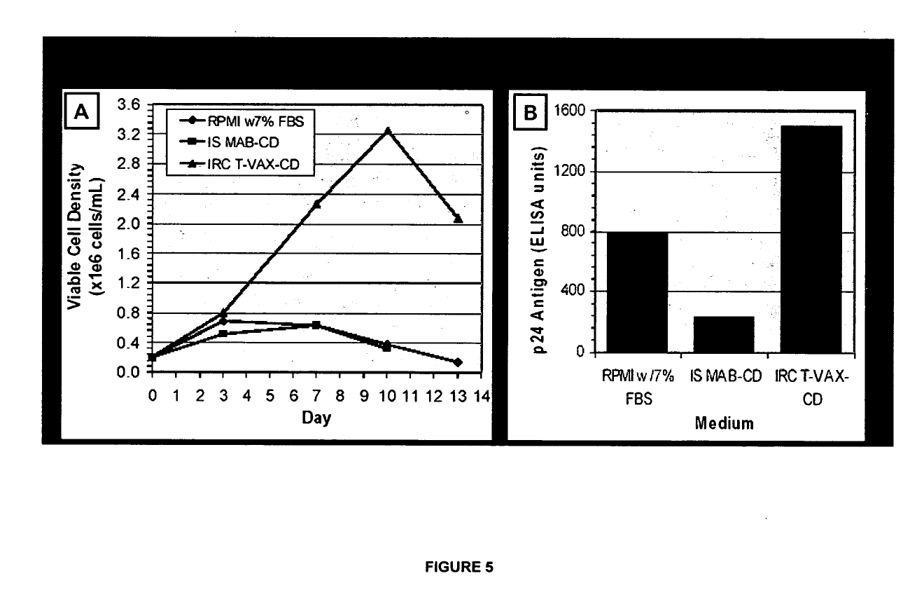

A 1200 L GMP lot of the final formula (named IRC T-VAX-CD) was produced and compared to the original production medium (RPMI-1640 with 7% FBS) and to the IS MAB-CD from which T-VAX-CD was derived in a set of roller bottles as shown in FIG. 5. The T-VAX-CD medium supported growth levels over three times as great as the other media. Virus production was double that obtained with RPMI-1640 and almost 6-fold greater than with IS MAB-CD. Downstream analysis of the purified virus derived from T-VAX-CD medium by several analytical techniques has shown that it is essentially equivalent to that produced in RPMI-1640.

In more detail, the analysis included the characterization of purified HIV-1 antigen prepared as described above. This allows comparison of HIV produced in protein free, serum free media to previously described methods of producing HIV in the presence of RPMI-1640 and serum to determine comparability of the methods and develop appropriate reference standards.

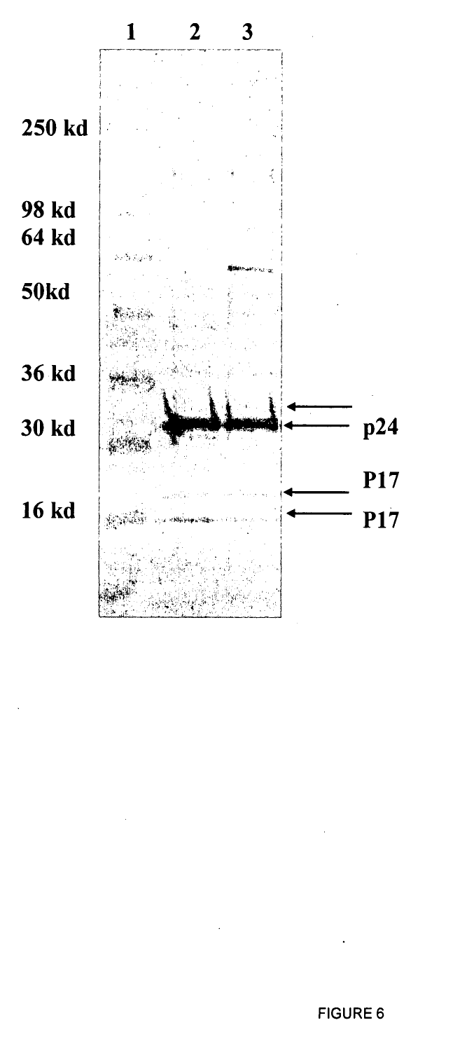

FIG. 6 shows analysis of purified HIV-1 antigen by Western immunoblotting. FIG. 6 shows immuno-staining profiles of a lot of HIV-1 antigen was produced from HIV-1 grown in the WAVE bioreactor system at the 5 liter scale and isolated using inactivation, filtration and anion exchange chromatography steps.

For Western blot characterization of HIV-1 antigen, the band size and migration distance was measured in comparison to the HIV-1 antigen derived from the standard process with HIV-1 produced in RPMI medium to determine if the antigen patterns are similar and/or equivalent. Analysis of the purified particles by Western blots utilized a standard HIV-1 antiserum isolated from HIV infected patients (New York Blood Center). The analytical emphasis is on monitoring mobility of selected bands. In this case, six bands meet the criteria for analysis, in that they are sharp, symmetrical, well resolved and therefore easily localized on a scan of the blot. These bands, in order of molecular weight as compared to standards, are the two p17 bands (p17.1, p17.2), p24, p39 gag intermediate (p17 & p24), p55 gag precursor polyprotein, and p66 reverse transcriptase.

FIG. 6 shows immunostaining profiles of a lot of HIV-1 antigen at a concentration of 1.0 micrograms/lane (lane 3). The staining profile for the RPMI derived reference standard lot of HIV-1 particles at a concentration of 1 microgram/lane is also shown (lane 2). Molecular weight markers are shown in lane 1. These results show that HIV-1 produced in protein free, serum free medium is comparable to that produced using standard procedures in RPMI in 7% fetal bovine serum.

Analysis of HIV-1 bulk antigen using SDS-PAGE was also performed. Analysis of the purified viral particles by Coomassie Blue stained SDS-PAGE gels is particularly valuable to evaluate the overall protein composition of the particles, since SDS can solubilize all components in the particle and Coomassie Blue stains all the protein bands separable by SDS-PAGE. SDS-PAGE analysis can be used to illustrate the comparability of the upstream manufacturing process used for producing the inactivated virus particles. Again, the strategy for consistency focused on overall similarity between the patterns, regarding the number of bands and their migration rather than strict quantification. The type of gel and buffer system used for analysis have been adjusted to provide high resolution of the lower molecular weight proteins (p24 and the two p17 bands) from each other as well as from the dye front (10-20% Tris-glycine gel system; Novex/Invitrogen catalogue E6135; Invitrogen, Carlsbad Calif.). This system allows monitoring of the mobility characteristics of these three important viral antigens. In optimizing separation of these bands, several bands at the higher molecular weight range become less resolved, as expected.

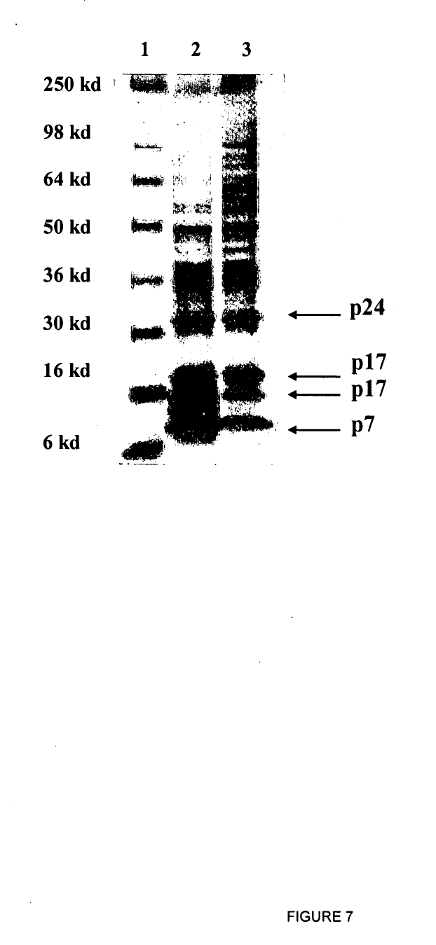

FIG. 7 shows analysis of purified HIV-1 antigen by SDS-PAGE. SDS-PAGE shows the staining profile for the reference standard lot of HIV-1 particles prepared by the standard “RPMI Process” (lane 2), and the HIV-1 antigen derived from protein free, serum free, chemically defined media utilizing the WAVE bioreactor system at the 5 liter scale (lane 3). These results show that purified HIV-1 antigen profiles are similar.

Specific activity analysis of p24 antigen, gp120 antigen, and residual host cell proteins of purified HIV-1 antigen was also performed. For the total protein assay, the protein concentration of HIV-1 antigen was determined using the bicinchoninic acid assay (BCA) developed by Pierce Co. (Rockford, Ill.). The BCA assay is a highly sensitive assay for the spectrophotometric determination of protein concentration for a given solution. The total protein assay is used as the basis for all specific activity analysis.

For the p24 antigen assay, a p24 ELISA was performed using a murine monoclonal antibody to HIV-1 coated onto microtiter plate wells. If HIV-1 p24 antigen is present in the sample, it will bind quantitatively to the antibody-coated wells. The bound antigen is then recognized by biotinylated antibodies to HIV, which reacts with conjugated streptavidin-horseradish peroxidase. Color develops from the reaction of the peroxidase with hydrogen peroxide in the presence of tetramethylbenzidine (TMB) substrate. The blue reaction is read at a visible wavelength over a fifteen-minute time period, and the kinetic rate of the reaction is measured. The rate of color development is directly proportional to the quantity of HIV-I p24 antigen present in the sample.

For the gp120 antigen assay, the gp120 Antigen Capture ELISA manufactured by Advanced Biotechnologies, Inc. (Columbia Md.) was used to detect and quantitate the amount of HIV-1 gp120 in HIV-1 antigen. The wells of the ELISA plate have been coated with a mouse monoclonal antibody which reacts with a number of divergent isolates of HIV-1. When gp120 is added, it forms an immune complex with the monoclonal antibody. Addition of peroxidase conjugated anti-HIV-1 antibody quantitatively detects the amount of bound HIV-1 gp120 within a given range.

For the host cell protein (HCP) assay, a sandwich ELISA protocol is used for quantitative determination of protein derived from HUT 78 host cells in HIV-1 antigen preparations.

The results of the specific activity analysis of p24 antigen, gp120 antigen and residual host cell proteins of purified HIV-1 antigen is shown in Table 3. The p24 analysis indicates nearly identical p24 antigen to total protein ratios for purified antigens derived from either chemically defined or RPMI media. Similarly, the gp120 analysis indicates nearly identical gp120 antigen to total protein ratios for antigens derived from either media. The HCP analysis also indicates nearly identical total host cell protein specific activity profiles for both antigens derived from either media.

TABLE 3
Specific Activity Analysis of p24 antigen, gp120 antigen,
and residual host cell proteins of purified HIV-1 antigen

| | Purified HIV-1 | Purified HIV-1 from PF/CD medium | | | | | |
|---|---|---|---|---|---|---|---|
| Assay | from RPMI process | Run 2 | Run 3 | Run 4 | Run 5 | Run 7 | Average |
| p24 | 15.4% | 10.4% | 14.2% | 17.7% | 16.8% | 16.8% | 15.2% |
| Antigen | | | | | | | |
| gp120 | 0.012% | 0.011% | 0.006% | — | 0.014% | — | 0.010% |
| Antigen | | | | | | | |
| Host Cell | 4.2% | 2.9% | 4.1% | 7.2% | 6.6% | — | 5.2% |
| Proteins | | | | | | | |

Additional analyses of HIV-1 antigen produced in chemically defined, serum free medium versus standard RPMI with serum can be performed. Electron microscopy analysis can be performed using complete transmission electron microscopy (TEM) analysis via negative staining. Protein free, chemically defined media derived antigen is evaluated by electron microscopy to show the expected characteristics of the virus particles. Specifically, the size and morphology of the electron dense stained material is evaluated for consistency with purified HIV-1. The micrographs can also be used to assess the degree of purity as shown by the lack of extraneous irregular shaped membrane fragments. Purified material from both processes are examined by electron microscopy to show the expected characteristics of the viral particles and lack of extraneous materials.

Residual DNA content can also be assessed. The presence of residual human DNA in Inactivated HIV-1 Antigen Drug Substance is determined by nucleic acid hybridization. Residual human DNA can be derived from the HUT-78 cells and can be used as another measure of purity. Several steps are performed for the DNA hybridization assay, which include: (1) extraction of DNA from contaminating protein in the test article, positive control spikes and negative controls. A positive control spike is made into the sample using the same genomic DNA used to make the probe for the assay; (2) preparation of standard curves; (3) denaturation of test article, negative and positive controls and standard curve DNA; (4) addition of test article, positive spikes, negative control, and standard controls to the membrane for hybridization; (5) preparation of labeled probe DNA; (6) hybridization of the labeled probe DNA to the test article, positive spikes, negative control, and standard DNA; and (7) visualization of the hybridization. Comparative levels of residual cellular DNA in both of the purified HIV-1 particle preparations are measured by a quantitative assay. The assay is based on a hybridization slot-blot analysis of residual cellular DNA using a genomic DNA probe isolated from an uninfected HuT78 cell line. The assay measures cellular DNA, not viral DNA. In this system, the detection and quantitation of the residual cellular DNA fragments are size independent. In other words, this assay provides an accurate measurement of the quantity of residual genomic DNA, but does not provide any information on its integrity. DNA analysis demonstrates comparable residual host cell DNA quantities for these antigens.

A reverse phase high performance liquid chromatography (RP-HPLC) profile, or other chromatographicic profiles, can also be determined to assess purity. HIV-1 antigen is analyzed using HPLC. The retention time and height of at least 6 well-characterized peaks in the sample are compared to a suitable HIV-1 reference standard. A representative HPLC tracing of the purified particles shows the complex nature of its protein components. The reverse phase HPLC profile is generated using conditions designed to yield optimal resolution of the complex protein mixture. Briefly, the HIV-1 antigen is solubilized and fractionated using a Vydac C4 reverse phase column using an Agilent HP1100 system (solvent A: 0.1% trifluoroacetic acid in water; solvent B: 0.1% trifluoroacetic acid in acetonitrile; solvent C, 0.1% trifluoroacetic acid in isopropanol. The profile demonstrates a minimum of 12 peaks that are partially resolved by this procedure. Fractions from this HPLC are collected and subjected to protein sequencing. Sequencing is performed to show the presence of gag proteins (p6, p7, p17, p24), β2 microglobulin (β2-m) and HLA Class II. The comparative patterns of the RPMI and protein free, chemically defined process are visually overlaid for direct comparison to show substantial equivalence. The RP-HPLC patterns thus also demonstrate comparability.

The HIV-1 antigen produced in chemically defined, protein and serum free media is also compared to standard RMPI prepared HIV-1 antigen in several immunological assays. Briefly, peripheral blood mononuclear cells (PBMCs) from blood samples drawn from drug naive HIV positive patients previously immunized with HIV-1 antigen produced in RPMI medium containing serum are frozen and then analyzed for induction of immune responses by ELISPOT, intracellular cytokine (ICC), and flow cytometry. Induction of these immune parameters are important to evaluate the potency of the vaccine to induce HIV specific immunity. For each in vitro assay, two identical sets of samples of the frozen PBMC's from the selected responding immunized patients are then stimulated under identical conditions as appropriate for each assay with equivalent mass amounts of the immunizing antigen purified from serum containing media (set 1) or the antigen purified from protein free media (set 2). Equivalent masses of p24 are used to determine the mass of antigen. These assays are well described in the art, and quantitate either IFN (gamma), interleukin-2 (IL2) or both made by various subtypes of CD4 T cells and CD8 T cells. Results from each assay are then evaluated for each assay, with the key comparisons being the magnitudes of response to the immunizing antigen purified from serum containing media to the responses seen to the antigen grown in protein free media. Equivalent responses between the two antigens strongly indicate that the immune cells induced by antigens in the immunizing antigen preparation can recognize the identical antigens in the antigen grown and isolated in protein free media. These assays indicate the antigens in the two preparations are immunologically equivalent.

Assays for INFγ are well known in the art (see for example, U.S. publication 2005/0196411). Briefly, single cell suspensions are prepared from spleens of the immunized mice by mincing and pressing through a sterile fine mesh nylon screen in RPMI 1640 (Hyclone, Logan, Utah). The splenocytes are purified by ficoll gradient centrifugation. CD4 and CD8 cells are isolated by magnetic bead depletion. 2×10⁷ cells are stained with 5 μg of either rabbit or rat anti-mouse CD4 or rabbit or rat anti-mouse CD8. Cells are incubated on ice for 30 minutes and washed with ice cold 2% Human AB serum in PBS. Pre-washed Dynabeads (DYNAL, Oslo, Norway) coated with goat anti-mouse IgG are added to the cell suspension and incubated at 4° C. for 20 minutes with constant mixing.

Purified CD4, CD8 and non-depleted splenocytes are resuspended in complete media (5% inactivated Human AB serum in RPMI 1640, Pen-strep, L-glutamine and 13-ME) at 5×10⁶ cells/ml and used for ELISPOT assay to enumerate the individual IFN-γ secreting cells. Briefly, 96 well nitrocellulose bottom microtiter plates (Millipore Co., Bedford, U.K.) are coated with 400 ngs per well of rabbit anti-mouse IFN-γ (Biosource, Camarillo, Calif.). After overnight incubation at 4° C., plates are washed with sterile phosphate buffered saline (PBS) and blocked with 5% human AB serum in RPMI 1640 containing pen-strep, L-glutamine and β-ME) for 1 hour at room temperature. Plates are washed with sterile PBS and 5×10⁵ per well of splenocytes (purified CD4, purified CD8 or non-depleted) are added in triplicate and incubated overnight at 37° C. and 5% CO₂. Cells are cultured with media, OVA (Chicken Egg Ovalbumin, Sigma-Aldrich, St. Louis, Mo.), native p24 or gp120-depleted HIV-1 antigen. CD4 purified and CD8 purified splenocytes are assayed in complete media containing 20 units/ml of recombinant rat IL-2 (Pharmingen, San Diego, Calif.).

After washing unbound cells, 400 ng per well of the polyclonal rabbit anti-mouse IFN-γ are added and incubated at room temperature for 2 hours, then washed and stained with goat anti-rabbit IgG biotin (Zymed, San Francisco, Calif.). After extensive washes with sterile PBS, avidin alkaline phosphatase complex (Sigma-Aldrich, St. Louis, Mo.) is added and incubated for another hour at room temperature. The spots are developed by adding chromogenic alkaline phosphate substrate (Sigma, St. Louis, Mo.), and the IFN-γ cells are counted using a dissection microscope (×40) with a highlight 3000 light source (Olympus, Lake Success, N.Y.).

Whole blood for use in the assays is collected from immunized patients by venipuncture in Vacutainer tubes containing EDTA (ethylenediaminetetraacetic acid) (Becton Dickinson, Rutherford, N.J.). Peripheral blood mononuclear cells (PBMC) are separated on lymphocyte separation medium (Organon Teknica, Durham, N.C.) and washed twice in phosphate-buffered saline (PBS; Organon Teknica), and the number of viable leukocytes is determined by trypan blue exclusion. Analyses are generally performed on freshly collected cells, but can be performed on frozen cells, as appropriate.

For immunophenotypic analyses, lymphocyte subsets are evaluated by flow cytometric analysis, using 50 ml of EDTA peripheral blood incubated for 30 min at 4° C. with fluorochrome-labeled monoclonal antibodies (CD4 R-phycoerythrin-Cyanine 5 Tandem-TC-; CD8 TC; CD45RA fluoroscein isothiocyanate (FITC), CD62L PE; CD45RO FITC; CD38 PE; CD28 PE; CD27 FITC; Caltag Laboratories, Inc., Burlingame, Calif.; CCR7PE mouse IgG2a isotype, R&D Systems, Minneapolis, Minn.). After incubation, erythrocyte lysis and fixation of marked cells is performed using the Immuno-Prep EPICS Kit (Coulter Electronics) and Q-prep Work Station (Coulter Electronics, Miami Lakes, Fla.).

For flow cytometric analysis, peripheral blood cells (PBMC's) are washed in PBS and stained for 30 min at 4° C. in the dark with the following antibodies: CD4 Cy5PE (mouse IgG2a isotype, Caltag); CD8 Cy5PE (mouse IgG2a isotype). Cells used for intracellular cytokine staining are washed and fixed in Reagent A solution (FIX & PERM cell permabilization Kits; Caltag) for 10 min at room temperature in the dark. The cells are washed once again in PBS and resuspended in Reagent B (FIX & PERM cell permabilization Kits; Caltag) with cytokine-(IL-2 PE; IFN(γ) PE, mouse IgG2a isotype; IL-10 FITC; Caltag Laboratories). The cells are then fixed in 1% paraformaldehyde in PBS. The frequency of antigen-specific CD4+ (or CD8+) T cells is determined by subtracting the percentage of IL2+ or IFN(γ), CD4+ (or CD8+) T cells from unstimulated PBMCs from the percentage of PBMCs stimulated with antigen.

Cytometric analyses are performed using an EPICS XL flow cytometer (Beckman-Coulter Inc., Miami, Fla.) equipped with a single 15 mW argon ion laser operating at 488 nm interfaced with 486 DX2 IBM computer (IBM, Cambridge, UK). For each analysis, 200,000 events are acquired. Green florescence from FITC (FL1) is collected through 525-nm bandpass filter and deep-red fluorescence from Cy5PE (FL4) is collected through 670-nm bandpass filter. Data are collected using linear amplifiers for forward and side scatter and logarithmic amplifiers for FL1 and FL4. Samples are first run using isotype controls or single fluorochrome-stained preparations for color compensation.

Antigen-specific responses are analyzed. For ELISPOT, ELISA and ICC assays, PBMC from REMUNE™ immunized patients, in which REMUNE™ was prepared from HIV grown in standard medium containing serum, are incubated for 18 hours in the presence/absence of: 1) HIV-1 whole killed antigen (10 μg/mL) made in serum containing media and identical to the antigen used to immunize the patients; 2) HIV-1 antigen made in protein free media; 3) native p24 (np24)(10 μg/mL) positive control or 4) buffer control. CD28 antibody (Clone 37407.111; R&D) is added during incubation (1 μg/well) to facilitate co-stimulation. For cytokine analyses, 10 μg/ml of Brefeldin A (Sigma, St. Louis, Mo.) is added to the cell cultures during the last 6 hours of stimulation to block protein secretion. Analyses are performed on frozen PMBC's. Commercial kits are available for such assays, and the manufacturer's recommended procedures are followed.

The comparative purity and equivalence of the HIV-1 viral antigen particles derived from the protein free, animal component free, chemically defined process compared to the RPMI process was clearly demonstrated by antigenic/immunological staining and by analysis of p24 antigen, gp120 antigen and residual host cell protein. The conclusion from these initial studies is that the chemical characteristics of these downstream antigen products are remarkably consistent considering the complexity of the viral particles.

### CONCLUSIONS

Human T cell lymphoma cell line HUT-78 (chronically infected with HIV) can be grown and reliably passaged in a chemically-defined culture medium, with cell densities exceeding 2e6 (2×10⁶) cells/mL.

The cell culture process in chemically defined, protein and serum free medium resulted in HIV-1 viral antigen yields that were improved at least two-fold over the serum-supplemented medium originally used for production through the process of screening and optimization.

The most significant factor in the improvement of virus yield was the utilization of a mixture of fructose and glucose as the carbon-source.

The HIV-1 purified antigen derived from the protein free, chemically defined media process is substantially equivalent to the HIV-1 antigen derived from RPMI with serum utilizing identical purification methods.

This optimization of the current cell culture process is being further evaluated in scale up, downstream process, and product consistency studies to enable more efficient full scale cGMP production of whole inactivated vaccines for investigational use in the treatment of HIV-1/AIDS.

Throughout this application various publications have been referenced. The disclosures of these publications in their entireties are hereby incorporated by reference in this application in order to more fully describe the state of the art to which this invention pertains. Although the invention has been described with reference to the exampled provided above, it should be understood that various modifications can be made without departing from the spirit of the invention.

What is claimed is:

1. A composition comprising human immunodeficiency virus (HIV) in the presence of protein-free, chemically defined medium.

2. The composition of claim 1, wherein the medium comprises fructose.

3. The composition of claim 2, wherein the medium comprises 6 grams/liter fructose.

4. The composition of claim 3, further comprising 2 grams/liter glucose.

5. The composition of claim 1, wherein said HIV is HIV-1.

6. A composition comprising human immunodeficiency virus (HIV) free of animal components.

7. The composition of claim 6, wherein said HIV is HIV-1.

8. The composition of claim 6, wherein said composition comprises whole inactivated HIV.

9. A method for producing HIV, comprising growing HUT-78 cells infected with HIV in a protein-free, chemically defined medium.

10. The method of claim 9, wherein the protein-free, chemically defined medium comprises fructose.

11. The method of claim 10, wherein the medium comprises 6 grams/liter fructose

12. The method of claim 11, further comprising 2 grams/liter glucose.

13. The method of claim 9, further comprising producing whole inactivated HIV from said HIV infected HUT-78 cells.

14. The method of claim 9, wherein said HIV is HIV-1.

15. A composition comprising human immunodeficiency virus (HIV) produced by the method of claim 9.
